(19) United States
(12) Patent Application Publication
(10) Pub. No.: US 2006/0288495 A1
(43) Pub. Date: Dec. 28, 2006

(54) System for and method of soft surface remediation

(76) Inventors: Michael M. Sawalski, Racine, WI (US); Jeffrey R. Johansen, Barrington Hills, IL (US)

Correspondence Address: S.C. JOHNSON & SON, INC., 1525 HOWE STREET, RACINE, WI, 53403-2236, US

(21) Appl. No.: 11/168,624

(22) Filed: Jun. 28, 2005

(51) Int. Cl. D06B 1/02

(52) U.S. Cl. 8/148; 8/149.2; 68/5 A

(57) ABSTRACT

The present invention is a system for and method of soft surface remediation, In particular, soft surface remediation refers to controlling contaminants, such as dust, dust mites, hair, and foul odor, from surfaces, such as upholstery, draperies, and curtains, in a home. The system of the present invention is based on a four-phase system of process mechanisms including dislodging the contaminants, displacing the contaminants, disposing of the contaminants, and disinfecting/freshening the contaminants. The four main process mechanisms are accomplished via the use of one or more technical mechanisms such as mechanical, electrostatic, acoustic, forced-air, and chemical. Further the technical mechanisms and process mechanisms can be represented by an overall system where combinations of both technical mechanisms and process mechanisms are utilized to accomplish soft surface remediation.

### BACKGROUND OF THE INVENTION

1. Field of the Invention

The present invention relates to the remediation of soft surfaces and, in particular, to soft surfaces, such as upholstery, draperies, and curtains, in the home.

2. Discussion of the Related Art

Increasingly, because of social trends, public safety issues, and the state of the economy, people prefer to spend more private time with their families inside the closed boundaries of their homes. As people spend more time in their homes, the home environment gets dirtier, because of the normal use and wear in the home, particularly in areas around the television, kitchen, and bathrooms. The use and wear in the home leads to an increase in airborne contaminants, such as dust, human skin flakes, pet hair, carpet fibers, upholstery, dust mite feces, hair, lint, allergens, mold/mildew, pollen, germs/bacteria, dust mites, particulates from electronics, household chemicals, radon, carbon monoxide, lead, asbestos, and smoke. Further, today, homes are designed and constructed to be as air tight as possible, for energy efficiency; therefore, the airborne contaminants tend to stay within the home and, furthermore, accumulate over time, as some contaminants, such as dust mites, feed and grow in population through other contaminants, such human skin flakes. Furthermore, it is known that more than 45% of homes have pets; however, over 80% of homes have pet hair found in them, which thereby demonstrates the persistence and transportability of airborne contaminants.

Many airborne contaminants in the home are in the form of “particulates” defined as smaller contaminants that are light enough to float in the air for a short duration. Particulates can be categorized further as light pet hair, skin flakes, dust mites, hazardous gases, dust, mold, and odors. Other contaminants that are larger and heavier than particulates include heavy pet hair, cereal crumbs, bread crumbs, and pebbles. “Contaminants” are defined herein to include all types of potential contaminants that can be found as airborne particulates.

The presence of airborne particulates can be measured as the concentration of weight of contaminants per volume of air (for example, X lbs per cubic foot). A high concentration of airborne particulates can cause allergic reaction, illness, and a generally undesirable environment in a home. The tendency of a well-sealed home to accrue elevated concentrations of allergens and illness-creating contaminants is called “sick house syndrome.” Thus, there is a need to remedy and/or prevent sick house syndrome by reducing the concentration of airborne particulates.

In general, it is impossible—and in fact not needed—to remove in the home “all” contaminants created by normal ecologic cycles (e.g., shedding skin flakes, dirt tracked into homes, pet shedding). Therefore, it is not required to completely eliminate the presence of contaminants; instead, what is needed is a mechanism to control (i.e., minimize) the introduction, generation, and retention of contaminants.

Typically, contaminants are present in three main areas of the home; these areas include the hard surfaces, the soft surfaces, and the air. The hard surfaces are “physically hard areas,” such as walls, tables, hard wood floors, kitchen cabinets, and certain types of smooth-surfaced furniture, such as leather furniture. The soft surfaces are physically soft surface areas formed of cloth, fibrous, or woven or textured surfaces, such as upholstery (sofas, chairs, and the like), mattresses, drapes, and carpet. Typically, the air is the open environment within the house. Contaminants vary in nature across the multiple surfaces. For example, dust can be present on hard surfaces, soft surfaces, and in the air, whereas carbon monoxide can only be present in the air. While numerous products currently exist for removing contaminants from hard surfaces and the air, there are few helpful solutions that address contaminants that settle on and in soft surfaces.

What is needed is an effective solution, to remediate soft surfaces in order to prevent sick house syndrome. Further, what is also needed is an effective solution to control (i.e., minimize) the introduction, generation, and retention of contaminants on and in soft surfaces. In addition, contaminants exist in a variety of shapes and sizes; therefore, what is also needed is a mechanism to control a wide variety of contaminants, such as dust, hair, microbials, organisms and odors, on soft surfaces.

A typical process of remediating a soft surface involves the four major steps of dislodging, displacing, disposing of, and disinfecting/freshening. Generally, dislodging the contaminant refers to freeing the contaminant from the surface. Generally, displacing the contaminant refers to moving the dislodged contaminant from its original location to a more desired location. Generally, disposing of the contaminant refers to disposing of or discarding the contaminant to an area away from the surface. Generally, disinfecting/freshening refers to (1) preventing or delaying the re-entry of a contaminant, (2) controlling, managing or reducing bad odor, (3) controlling, managing or reducing organisms that may act as or produce a contaminant (such as dust mites, mold, or mildew) and/or (4) adding a positive or pleasant scent.

For example, Windex® Original, a product sold by SC Johnson & Son, Inc., is a liquid with a key ingredient, Ammonia-D® that can be used to clean hard surfaces, such as glass, mirrors, chrome, plastic, vinyl, and stainless steel. In a typical mode of operation, a user sprays Windex® onto a hard surface, in order to dislodge the contaminants from the surface, and uses a cloth to soak up the Windex® and, thus, dispose of the contaminants from the hard surface. However, a typical user would not use Windex® on a soft surface, because doing so would physically impact the soft surface, by soaking the soft surface and redistributing or repositioning the contaminant. Not only would a Windex®-soaked soft surface be less appealing, but the residual moisture would, in fact, attract additional airborne contaminants and/or encourage the growth of the organic contaminants that the user desires to remove.

Another example of removing contaminants from soft surface, without physically impacting the soft surface, is a “lint brush” of a typical design, which includes a roller element on a short handle. The device rolls on a vertical axis parallel with the handle and provides an outward-facing, mild adhesive surface that is similar to masking tape, in order to attract and hold (dislodge, displace, and dispose of) contaminants.

However, the amount of contaminants dislodged and displaced by the lint brush is directly related to the size of the “contact area” (the area of the lint brush that contacts the surface at any given moment). Contaminants not completely reached by the contact area will not be dislodged and disposed of by the lint brush, which thereby makes the lint brush inefficient and, in many cases ineffective. What is needed is a mechanism to control a variety of contaminants by dislodging the contaminants, without harming the soft surface. What is also needed is a mechanism to control a variety of contaminants by disposing of the contaminants, without harming the surface.

Another product, PlugIns®, also sold by SC Johnson & Son, Inc., is an electric air freshener that emits fragrance oil into the air of the home, in order to remediate the air from odor caused by contaminants through the use of positive fragrancing. In a typical mode of operation, electricity provides energy to heat the fragrance oil, which causes the oil to evaporate and spread into the surrounding air and further freshen the surrounding air from contaminants that cause malodor. However, the fragrance oil and device cannot be used to remediate or freshen soft surfaces. What is needed therefore is a mechanism to control a variety of contaminants, by disinfecting/freshening the contaminants, without harming the surface.

Yet another product is described in U.S. Patent Application US20030070251, entitled “Vacuum Cleaner” (hereinafter the “'251 patent application” wherein a vacuum cleaner is described, including: (1) a surface cleaning head that defines an inlet, (2) a container for collecting dirt and dust and a primary passageway connecting the inlet in the cleaning head to the container, and (3) a suction mechanism for drawing dirt and dust in through the inlet in the cleaning head, along the primary passageway, and into the container. One or more secondary inlets are provided on a surface of the vacuum cleaner that is remote from the inlet in the cleaning head, in order to drawn in airborne dirt and dust.

The vacuum cleaner of the '251 patent application utilizes a vacuum head to dislodge the contaminants, a suction mechanism to displace the contaminants, and a container for collecting dirt and dust as a mechanism to dispose of the contaminants. However, the suction mechanism to displace the contaminants of the '251 patent application could cause damage to soft and delicate surfaces, in particular, to surfaces such as drapes and fine upholstery fabric, because of the excessive suction force. Therefore, the vacuum cleaner of the '251 patent is more effective on hard surfaces or “semi-hard” surfaces, such as a sturdy carpet or rug, than on soft surfaces. What is needed is a mechanism to control a variety of contaminants, by dislodging, displacing and, further, disposing of contaminants, without harming the soft surface.

U.S. Pat. No. 5,968,204, entitled, “Article for cleaning surfaces” (hereinafter the “'204 patent”) describes sheets capable of developing a positive electrostatic charge that are used for a variety of surface cleaning operations. Polyester fabric, non-woven and chemically bonded with an acrylic latex, is used to dust surfaces and to clean clothes, furniture, and carpets. A light solvent, such as isopropanol, can be used with the sheets to loosen gummy soils. In one mode, the sheets are used in a hot air clothes dryer to remove soils and detritus from garments. Processes for cleaning a variety of fabric and hard surfaces are conducted by using the sheets.

While the '204 patent demonstrates a mechanism to dislodge, displace, and dispose of soils and detritus from garments (i.e., soft surfaces), the '204 patent does not demonstrate a mechanism to disinfect the garment. The simultaneous dislodging, displacing, disposing of, and disinfecting could be highly beneficial to the user. Furthermore, as mentioned above, a large variety of contaminants can exist on a soft surface, and the electrostatic charge mechanism of the '204 patent does not demonstrate a way to remove most types of contaminants. For example, sand, being heavier than normal dust, needs to be shaken loose from the fibers within the fabric. Also even dust that has settled and is not on the surface needs to be dislodged from the fibers to be successfully removed. What is needed is a mechanism to control a variety of contaminants, by simultaneously dislodging, displacing, disposing of, and disinfecting/freshening the contaminants, without harming the surface and in some cases with minimum surface impact.

The disclosures of all of the below-referenced prior United States patents, and applications, in their entireties are hereby expressly incorporated by reference into the present application for purposes including, but not limited to, indicating the background of the present invention and illustrating the state of the art.

U.S. Pat. No. 6,746,166, entitled “Apparatus for cleaning a surface,” assigned to Art Center College of Design (Los Angeles, Calif.), describes an apparatus for efficiently cleaning stains and extracting cleaning fluid from surfaces such as carpets and upholstery without requiring electrical power. The invention eliminates the inconvenience of retrieving, filling with cleaning fluid, and plugging a deep cleaner into an electrical outlet in order to remove a small spot from a carpet. In addition to the scrubbing and fluid extracting capabilities, embodiments of the invention include a sprayer for applying cleaning fluid to stains. The sprayer may receive fluid from an attached refillable reservoir of cleaning solution, for example. During scrubbing, the top of a pump actuator provides resting place for the heel of a user's palm. The pump actuator may be locked down when scrubbing and unlocked for pumping to suck up fluid. A piston in a chamber provides the suction force for pulling fluid up through tubules, which may be interspersed between bristle tufts, past check valves and into a waste reservoir. A downward force on the piston provides the suction thereby assuring that the tubules are in contact with the surface during suction. The waste reservoir may be dumped via a plug in the waste reservoir.

U.S. Pat. No. 5,515,569, entitled “Handbrush especially for cleaning carpets and upholstery,” assigned to Monti-Werkzeuge GmbH (Konigswinter, DE), describes a hand brush for upholstery and carpets can have its brush body formed with at least one elastically bendable bristle band colinear with a handle and provided with bristles which can project forwardly on the band.

U.S. Pat. No. 4,961,271, entitled “Apparatus for treating furniture,” invented by Christopher S. Butler (Dallas, Tex.), describes an apparatus for treating furniture that has been wet cleaned or damaged by water or smoke. The apparatus is a tent having a floor, a plurality of sides, and a top, forming an enclosed chamber. The furniture is placed on the floor within the sides, and the top is attached to the sides. Air or ozone is then blown into the chamber to inflate the tent. A plurality of holes in the top of the tent or exhaust boots in the sides of the tent allow the air or ozone to escape from the chamber at a selected rate to maintain the inflation of the tent.

U.S. Pat. No. 4,703,538, entitled “Cleaning tool,” invented by Catherine A. Silverstrone (Bedford, Mass.), describes a cleaning tool which is especially suited for picking up dirt, lint and the like from rugs, floors, upholstered furniture and other surfaces. The cleaning tool comprises, in one embodiment of the invention, an elongated stick shaped handle, a pair of legs extending outward from one end of the handle, a cylindrically shaped cleaning element having an external surface made of Velcro, a disc with teeth on its periphery fixedly mounted at each end of the roller shaped cleaning element and a disc with slots fixedly mounted at the outer end of each leg. Each slotted disc is adapted to fixedly and matingly engage one of the discs at the ends of the cleaning element. In use, the cleaning tool is pushed over the surface to be cleaned.

U.S. Pat. No. 4,671,567, entitled “Upholstered clean room seat,” assigned to The Jasper Corporation (Haskins, Ohio), describes that in order to assist in complying with strict Federal standards of cleanliness for industrial clean rooms, a sealed upholstered seat is placed in communication with an underlying relatively shallow breather bag which is retained by a seat pan attached to a rigid panel of the seat. The breather bag receives air from the upholstered seat when the seat is compressed and returns the same air to the seat as the seat expands. The escape of particulate matter from the seat into the atmosphere during usage of the seat is eliminated. A seat standard is received through an opening in the pan and passes between two spaced portions of the breather bag. The top of the standard is welded between the side walls of a channel member having side flanges which are attached to the bottom of the seat panel.

World Intellectual Property Organization Application No. WO9708983, entitled “A vacuum cleaner,” assigned to Magiview Pty. Ltd. (Australia), describes a vacuum cleaner head (10) comprising a curved body (15) about which fluid can circulate, the curved body having a lower portion (17) which in use is adapted to be adjacent the area to be vacuumed. The lower portion is dimensioned to provide a lower pressure surface. The curved body also has an upper portion (18) dimensioned to provide a higher pressure surface. A fluid acceleration means (21) accelerates the fluid about the curved body. A fluid stream splitter (31) is in the upper portion of the curved body to split the fluid into a recirculated portion which continues to move about the curved body. A waste portion passes waste into a dust collecting chamber (32) and thus exhausts it therefrom.

World Intellectual Property Organization Application No. W00187113, entitled “Device for taking care of objects, comprises a scraper element,” invented by Jordan Gustav (Germany). This application describes a device for taking care of objects, in particular for brushing and cleaning carpets, upholstered furniture or similar and/or for grooming the hair of a living thing, using bristles (2), that are arranged on a support (1). According to the invention, a scraper element (6) is provided at least partially between the bristles (2) to scrape off impurities, in particular hairs, lint, etc.

### SUMMARY OF THE INVENTION

By way of summary, it is therefore an aspect of the invention to provide an effective solution to remediate soft surfaces, in order to prevent sick house syndrome.

It is another aspect of this invention to provide an effective solution to control (i.e., minimize) the introduction, generation, and retention of contaminants on soft surfaces.

It is yet another aspect of this invention to provide a mechanism to control a variety of contaminants, such as dust, hair, microbials, and odors, on soft surfaces.

It is yet another aspect of this invention to provide a mechanism to control a variety of contaminants, by dislodging contaminants, without harming the soft surface.

It is yet another aspect of this invention to provide a mechanism to control a variety of contaminants, by displacing the contaminants, without harming the surface.

It is yet another aspect of this invention to provide a mechanism to control a variety of contaminants, by disposing of the contaminants, without harming the surface.

It is yet another aspect of this invention to provide a mechanism to control a variety of contaminants, by disinfecting/freshening the contaminants without harming the surface.

It is yet another aspect of this invention to provide a mechanism to control a variety of contaminants, by simultaneously dislodging, displacing, disposing of, and disinfecting/freshening the contaminants, without harming the surface.

These, and other aspects and objects of the present invention will be better appreciated and understood when considered in conjunction with the following description and the accompanying drawings. It should be understood, however, that the following description, while indicating preferred embodiments of the present invention, is given by way of illustration and not of limitation. Many changes and modifications may be made within the scope of the present invention without departing from the spirit thereof, and the invention includes all such modifications.

### BRIEF DESCRIPTION OF THE DRAWINGS

A clear conception of the advantages and features constituting the present invention, and of the construction and operation of typical mechanisms provided with the present invention, will become more readily apparent by referring to the exemplary, and therefore non-limiting, embodiments illustrated in the drawings accompanying and forming a part of this specification, wherein like reference numerals designate the same elements in the several views, and in which:

FIG. 1 illustrates a soft surface remediation system in accordance with the present invention.

FIG. 2 illustrates a soft surface remediation system, enabled through a mechanical technical means in accordance with the present invention.

FIG. 3 illustrates a soft surface remediation system, enabled through an electrostatic technical means in accordance with the present invention.

FIG. 4 illustrates a soft surface remediation system, enabled through an acoustic technical means in accordance with the present invention.

FIG. 5 illustrates a soft surface remediation system, enabled through a forced-air technical means in accordance with the present invention.

FIG. 6 illustrates a soft surface remediation system, enabled through a chemical technical means in accordance with the present invention.

FIG. 7 illustrates a method of implementing a soft surface remediation system in accordance with the present invention.

FIG. 8 illustrates a combination system that includes a series of process mechanisms, a series of technical mechanisms, an option A, an option B, and an option C in accordance with the present invention.

In describing the preferred embodiments of the invention which is illustrated in the drawings, specific terminology will be resorted to for the sake of clarity. However, it is not intended that the invention be limited to the specific terms so selected and it is to be understood that each specific term includes all technical equivalents which operate in a similar manner to accomplish a similar purpose. For example, the word “connected” or terms similar thereto are often used. They are not limited to direct connection but include connection through other elements where such connection is recognized as being equivalent by those skilled in the art. Further, for the purposes of this disclosure, the term “cleaning” or “cleaned” is broadly expanded to include operations associated with soft surface remediation (SSR). The materials used for further cleaning may include cleaning chemicals, odor eliminators, stain removal, fabric protectors, fresheners, and disinfectants all of which may be in the form of liquids, gases, solids, gels, substrates and/or powders or combinations thereof.

### DESCRIPTION OF PREFERRED EMBODIMENTS

The present invention and the various features and advantageous details thereof are explained more fully with reference to the non-limiting embodiments described in detail in the following description.

1. System Overview

The present invention is a system or a set of systems for and method of soft surface remediation. The system of the present invention is based on four phases of process mechanisms including dislodging, displacing, disposing of, and disinfecting/freshening, which is accomplished preferably via a system that utilizes at least one (or a combination) of technology mechanisms that include mechanical, acoustic, electrostatic, forced air, and chemical mechanisms.

2. Detailed Description of the Invention

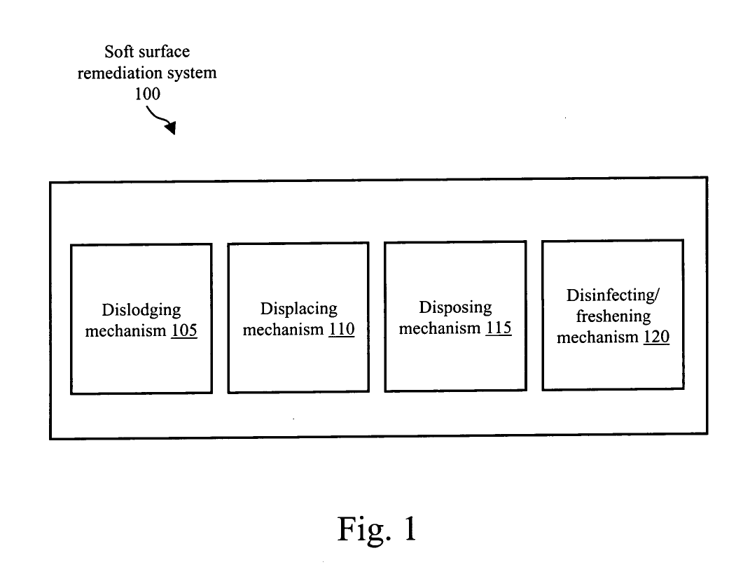

In a first preferred embodiment, the invention is a generalized soft surface remediation system that includes the four phases of process mechanisms including dislodging, displacing, disposing of, and disinfecting/freshening. FIG. 1 illustrates a functional block diagram of a soft surface remediation system 100 that includes a dislodging mechanism 105, a displacing mechanism 110, a disposing mechanism 115, and a disinfecting/freshening mechanism 120. Soft surface remediation system 100 represents the base system from which the other system configurations described herein for soft surface remediation are derived.

Dislodging mechanism 105 refers to the mechanism of freeing contaminants (not shown) from or near a soft surface (not shown). Dislodging mechanism 105 is the tool and/or force and/or field (such as electromagnetic) that disengages contaminants from a soft surface, which, generally, requires sufficient force to overcome the force of adhesion or attraction of the contaminant to the soft surface. Examples of dislodging mechanism 105 may include mechanical “slapping” tools, electrostatic attraction materials, air-flow tools, and the like.

Displacing mechanism 110 refers to the moving of contaminants after they have been freed from or near a soft surface and, often, one apparatus can provide the function of both the dislodging mechanism 105 and the displacing mechanism 110. Examples of displacing mechanism 110 include similar tools to dislodging mechanism 105 but provide sufficient force to move contaminants away from the soft surface being remediated.

Disposing mechanism 115 refers to the capture, removal, and disposal of contaminants after they are freed and moved from or near a soft surface. Examples of disposing mechanism 115 include filters, wovens, non-wovens, contaminant-catching cups or containers, sticky tape, fly paper, contaminant-attractive gels, and the like.

Disinfecting/freshening mechanism 120 refers to application of a treatment to control dust mites, bacteria, mold, and the like or, alternatively, to remove odors or otherwise improve the scent or perceived “freshness” of the soft surface. Because there is a range of need from biocide to aesthetics (odor and scent), the term “disinfect” is taken more loosely than strict definition of biocidal capacity. Examples of disinfecting/freshening mechanism 120 include chemistries (sprays, liquids, foams, aerosols) or technologies (e.g., ionic wind, gas injection, etc.) that provide such results as odor removal, biocidal qualities, impairment to organic growth, or positive fragrancing.

In another preferred embodiment, a protect and/or renew mechanism may be added. Such a mechanism may be a substance like SCOTCHGUARD® which adds a layer of protectant to the soft surface, or provides some coating or barrier that will cause the contaminants not to adhere to the soft surface.

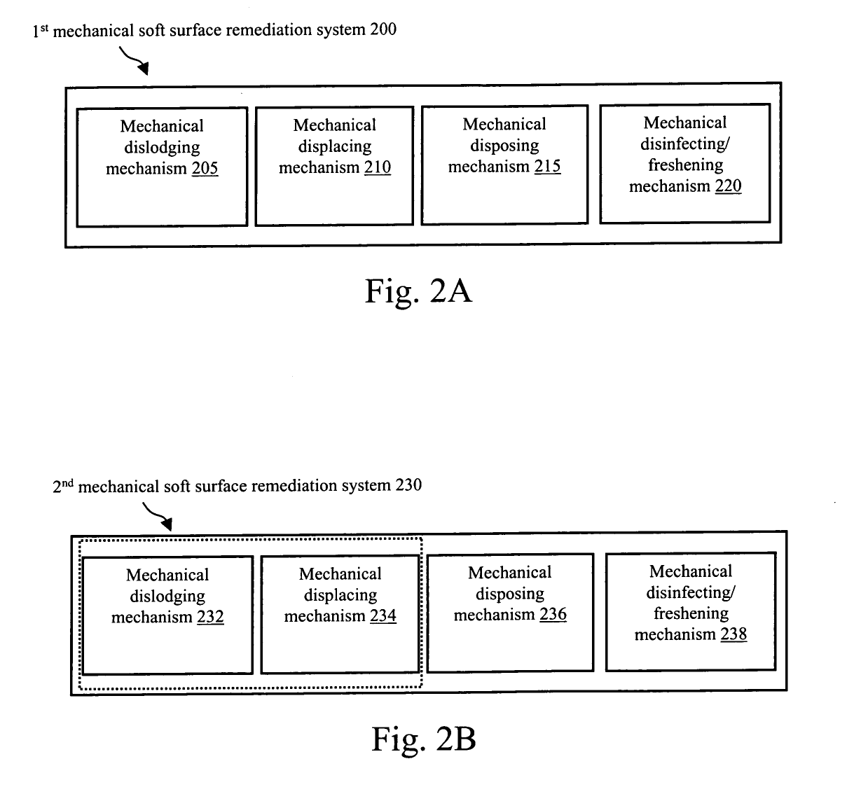

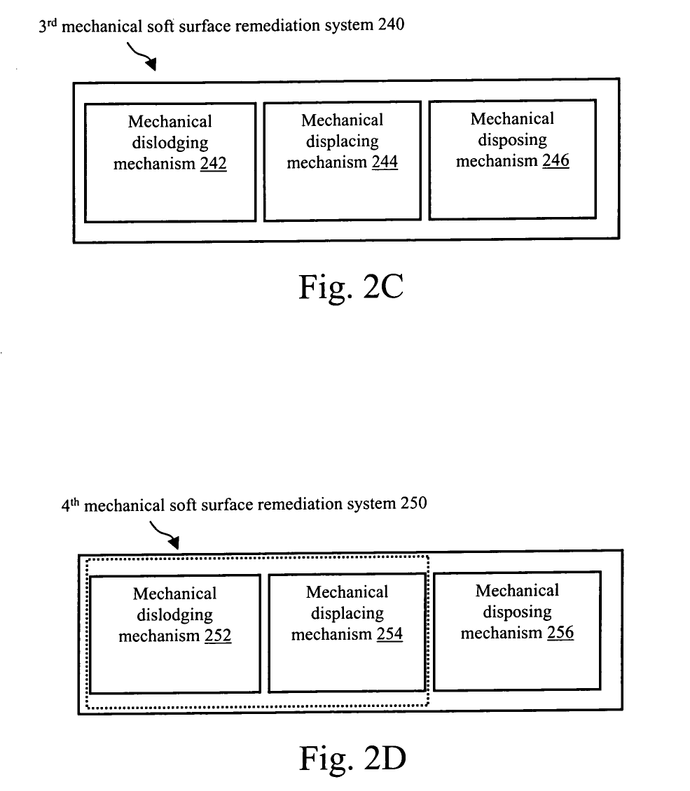

Multiple technical mechanisms exist for dislodging mechanism 105, displacing mechanism 110, disposing mechanism 115, and disinfecting/freshening mechanism 120, to remediate a soft surface. In one example, a mechanical approach includes a tool that utilizes mechanical (i.e., physical) force, in combination with a collection tray, in order for dislodging mechanism 105, displacing mechanism 110, and disposing mechanism 115 to control contaminants and remediate a soft surface. In another example, an electrostatic approach includes an electric charge, in order for displacing mechanism 110 to control contaminants and remediate a soft surface. In another example, an acoustic approach, such as a resonant bass tone, is used, in order for dislodging mechanism 105 and displacing mechanism 110 to control contaminants and remediate a soft surface. In another example, an air approach, such as an air fan, in conjunction with an air filter and sanitization fluid, is used for dislodging mechanism 105, displacing mechanism 110, disposing mechanism 115, and disinfecting/freshening mechanism 120 to control contaminants and remediate a soft surface. In another example, a chemical approach is used, in order for disinfecting/freshening mechanism 120 to control contaminants and remediate a soft surface. The above-mentioned technical mechanisms are described in more detail in reference to FIGS. 2, 3, 4, and 5.

In summary, multiple technical mechanisms, chosen to avoid harming the soft surface, are utilized to achieve dislodging mechanism 105, displacing mechanism 110, disposing mechanism 115, and disinfecting/freshening mechanism 120, in order to remediate soft surfaces and, thus, prevent sick house syndrome and control (i.e., minimize) the introduction, generation, and retention of contaminants on soft surfaces. The combination of these technical mechanisms is described in greater detail as the final embodiment of the invention.

In a second embodiment, to accomplish the four phases of dislodging, displacing, disposing of, and disinfecting/freshening, a mechanically based system is provided. FIG. 2A illustrates a first mechanical soft surface remediation system 200 that includes a mechanical dislodging mechanism 205, a mechanical displacing mechanism 210, a mechanical disposing mechanism 215, and a mechanical disinfecting/freshening mechanism 220. Each of these four mechanisms is presumed to be a separate and distinct apparatus within the system.

Mechanical dislodging mechanism 205, mechanical displacing mechanism 210, mechanical disposing mechanism 215, and mechanical disinfecting/freshening mechanism 220 are defined similarly as dislodging mechanism 105, displacing mechanism 110, disposing mechanism 115, and disinfecting/freshening mechanism 120, respectively, of soft surface remediation system 100, as described in FIG. 1.

Mechanical dislodging mechanism 205 consists of a tool utilizing mechanical energy, such as impact, scraping, brushing and pressure to accomplish the dislodging function. Examples of mechanical dislodging mechanism 205 include a slapping tool, a beating tool, a pounding tool, a brushing tool, a shaking tool, or a rotating or rotational physical force tool.

Similar types of tools, such as slapping tool, a brushing tool, a pounding tool, a shaking tool, or a rotational physical force tool, are appropriate for mechanical displacing mechanism 210. In the case of displacing, the force or energy required to accomplish displacement may be greater than that required for dislodging. The mechanical energy that supplies mechanical displacing mechanism 210 may be provided in various physical or electrical formats (e.g. a wind-up mechanism, an AC/DC electricity source, etc).

Mechanical disposing mechanism 215 consists of an apparatus that collects and disposes of contaminants. Examples of mechanical disposing mechanism 215 are high-efficiency particulate air (HEPA) filters, such as those used in a vacuum cleaner, cyclone cups, cloth filters, and the like. High airflow (HAF) filters may also be used.

Mechanical disinfecting/freshening mechanism 220 is more limited in the forms that successfully accomplish disinfecting/freshening. Mechanical designs provide the source of energy to deliver either a disinfecting or freshening effect to the soft surface. Examples include heat, for example using a heating element such as an electrically heated coil to apply hot, moisturized air (e.g., steam) onto a soft surface. In another example, a mechanical dispensing system may atomize a chemical formulation (not shown) upon the soft surface. Another example would be to take the ambient hot air around the motor and use it as a catalyst to begin a chemical process of disenfectancy/fragrancing. On other example, is the injection of air or active gas.

Additional forms of all four process mechanisms: mechanical dislodging mechanism 205, mechanical displacing mechanism 210, mechanical disposing mechanism 215, and mechanical disinfecting/freshening mechanism 220 may be suggested easily by those skilled in the art.

In operation, a soft surface is remediated by using mechanical dislodging mechanism 205 to deliver sufficient energy to the soft surface to force contaminants to release from the soft surface. Then mechanical displacing mechanism 210 provides the energy to move contaminants away from the soft surface, where mechanical disposing mechanism 215 collects contaminants and precludes the contaminants from being released or resettling upon the soft surface. Finally, mechanical disinfecting/freshening mechanism 220 preferably provides energy to kill contaminants (in whole or in part) on a soft surface.

Thus first mechanical soft surface remediation system 200 provides a mechanism to control a variety of contaminants, by simultaneously dislodging, displacing, disposing of, and disinfecting/freshening the contaminants, without harming the soft surface.

In a second example shown in FIG. 2B, second mechanical soft surface remediation system 230 includes mechanical dislodging mechanism 232, mechanical displacing mechanism 234, mechanical disposing mechanism 236, mechanical disinfecting/freshening mechanism 238. In this case, mechanical dislodging mechanism 232 is combined with mechanical displacing mechanism 234 into a single apparatus. In one design, second mechanical soft surface remediation system 230 houses a set of slapping bars that perform the function of both mechanical dislodging mechanism 232 and mechanical displacing mechanism 234, i.e. the action of the slapping bars dislodges contaminants and provides sufficient force to displace and move the contaminants away from the soft surface. A woven filter, or like filtration device, acts as mechanical disposing mechanism 236 and a mechanical pump spray device provides the function of mechanical disinfecting/freshening mechanism 238. Thus second mechanical soft surface remediation system 230 can contain all four-process mechanisms in a combined element configuration.

In a third example shown in FIG. 2C, third mechanical soft surface remediation system 240 includes mechanical dislodging mechanism 242, mechanical displacing mechanism 244, and mechanical disposing mechanism 246. In this case, a mechanical disinfecting/freshening mechanism has been omitted and is not included in the design of third mechanical soft surface remediation system 240. Examples of mechanical dislodging mechanism 242, mechanical displacing mechanism 244, and mechanical disposing mechanism 246 are identical to those described in first mechanical soft surface system 240. Thus third mechanical soft surface remediation system 240 can provide for less than all four-process mechanisms in its design.

In a fourth example shown in FIG. 2D, fourth mechanical soft surface remediation system 250 includes mechanical dislodging mechanism 252, mechanical displacing mechanism 254, mechanical disposing mechanism 256, and mechanical disinfecting/freshening mechanism 258. In this case, a mechanical disinfecting/freshening mechanism has been omitted and is not included in the design of fourth mechanical soft surface remediation system 240, and also mechanical dislodging mechanism 252 is combined with mechanical displacing mechanism 254 into a single apparatus. The examples cited in the second and third examples above also apply to this example. Thus fourth mechanical soft surface remediation system 250 can provide for less than all four-process mechanisms and also combine process mechanisms in its design.

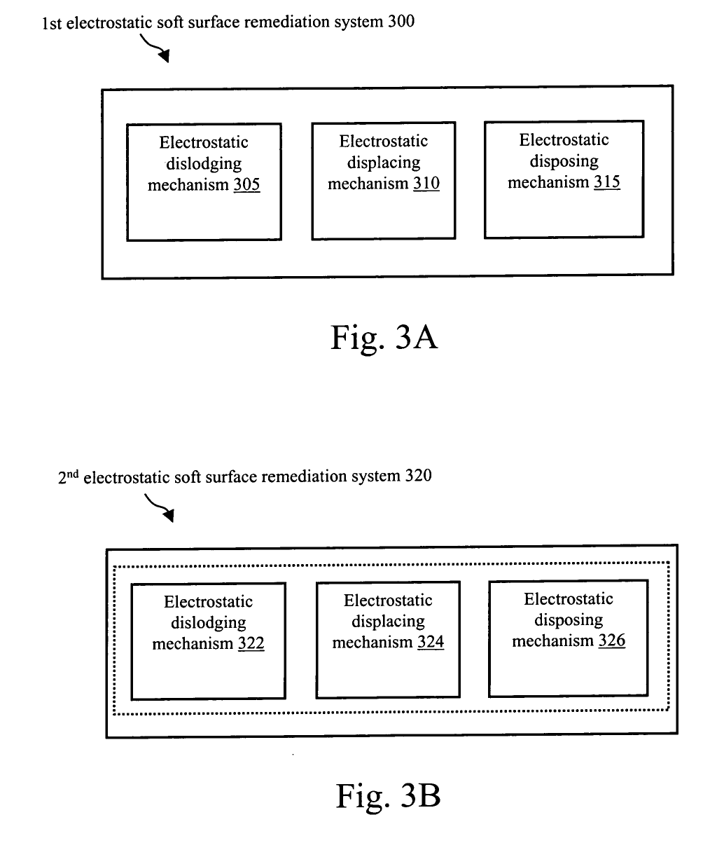

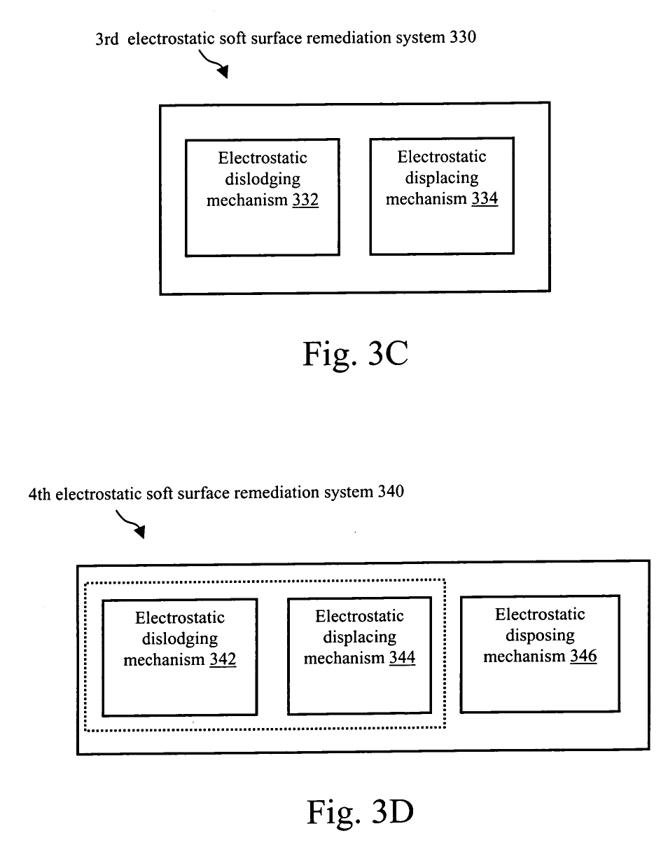

In a third embodiment, an electrostatically based system is provided. Note that in this embodiment, no disinfecting/freshening mechanism is included. As electrostatic mechanisms cannot be used for disinfecting/freshening, another process mechanism from the other types described herein must be used to complete four-phase remediation. FIG. 3 illustrates a first electrostatic soft surface remediation system 300 that includes an electrostatic dislodging mechanism 305, an electrostatic displacing mechanism 310, and an electrostatic disposing mechanism 315.

Electrostatic dislodging mechanism 305, electrostatic displacing mechanism 310, and electrostatic disposing mechanism 315 are defined similarly as dislodging mechanism 105, displacing mechanism 110, and disposing mechanism 115, as described in FIG. 1.

Electrostatic dislodging mechanism 305 is a tool that utilizes electrostatic energy, such as static energy (related to the magnetic field created by the movement of charged electrons along a fixed path) Examples of electrostatic dislodging mechanism 305 include an electric generator or a charged battery that stores and releases an electric charge upon use.

Similar types of tools as described above are also appropriate examples for electrostatic displacing mechanism 310. The force applied to dislodge contaminants may be sufficient to also displace contaminants from a soft surface.

Electrostatic disposing mechanism 315 is a tool that utilizes electrostatic energy to collect and dispose of contaminants. For example, Swiffer®, a product sold by Procter & Gamble, is a disposable electrostatic cloth used to capture dirt, dust, and hair¹, this could be modified to be an electrostatic disposing mechanism 315.
¹http://www.pg.com/product_card/brand_overview.jhtml?brand=swiffer&category=Household+Cleaners&brandImage=%2Fcontent%2Fimage%2Fbrand_logos%2Fswiffer.jpg

Additional forms of all three process mechanisms: electrostatic dislodging mechanism 305, electrostatic displacing mechanism 310, and electrostatic disposing mechanism 315 may be suggested easily by those skilled in the art.

In operation, a soft surface is remediated by using electrostatic dislodging mechanism 305 to deliver sufficient energy to the soft surface to force contaminants to release from the soft surface. Then electrostatic displacing mechanism 310 provides the energy to move contaminants away from the soft surface, where electrostatic disposing mechanism 315 collects contaminants and precludes the contaminants from being released or resettling upon the soft surface. If desired, another process mechanism needs to be utilized to accomplish the step of disinfecting/freshening in first electrostatic soft surface remediation system 300.

Thus first electrostatic soft surface remediation system 300 provides a mechanism to control a variety of contaminants, by simultaneously dislodging, displacing, and disposing of the contaminants, without harming the soft surface.

In a second example shown in FIG. 3B, second electrostatic soft surface remediation system 320 includes electrostatic dislodging mechanism 322, electrostatic displacing mechanism 324, and electrostatic disposing mechanism 326. In this case, electrostatic dislodging mechanism 232 is combined with both electrostatic displacing mechanism 234 and electrostatic disposing mechanism 326 into a single apparatus.

In one design, second electrostatic soft surface remediation system 320 is a single apparatus of a woven or non-woven material that generate electrostatic energy via rubbing on a surface. This apparatus can satisfy the function of dislodging mechanism 322, electrostatic displacing mechanism 324, and electrostatic disposing mechanism 326. Thus second electrostatic soft surface remediation system 320 can combine all three possible process mechanisms in a combined element configuration.

In a third example shown in FIG. 3C, third electrostatic soft surface remediation system 330 includes electrostatic dislodging mechanism 332 and electrostatic displacing mechanism 334. In this case, an electrostatic disposing mechanism and an electrostatic disinfecting/freshening mechanism has been omitted, and is not included in the design of third electrostatic soft surface remediation system 330. Examples of electrostatic dislodging mechanism 332 and electrostatic displacing mechanism 324 are identical to those described in first electrostatic soft surface system 300. Thus third electrostatic soft surface remediation system 330 can provide for less than all four process mechanisms in its design.

In a fourth example shown in FIG. 3D, fourth electrostatic soft surface remediation system 340 includes electrostatic dislodging mechanism 342, electrostatic displacing mechanism 344, electrostatic disposing mechanism 346. In this case, an electrostatic disinfecting/freshening mechanism has been omitted and is not included in the design of fourth electrostatic soft surface remediation system 340, and also electrostatic dislodging mechanism 342 is combined with electrostatic displacing mechanism 344 into a single apparatus, such as a charge generator that creates an electric field which imparts a charge of a single polarity to the surface and the contaminants contained therein. The field then reverses, thereby attracting the charged contaminants from the soft surface. The examples cited in the second and third examples above also apply to this example. Thus fourth electrostatic soft surface remediation system 340 can provide for less than all four-process mechanisms and also combine process mechanisms in its design.

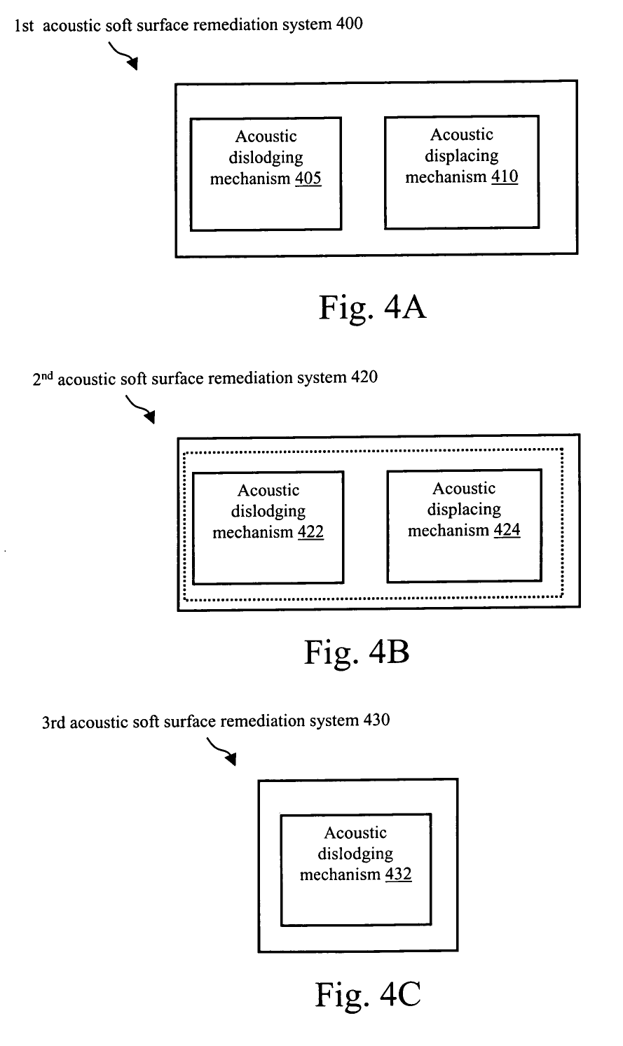

In a fourth embodiment, to accomplish two of the four phases of dislodging, displacing, disposing of, and disinfecting/freshening, an acoustically based system is provided. As acoustic mechanisms cannot be used for disposing of, or disinfecting/freshening, another mechanism (or mechanisms) from the other types described herein must be used to complete four-phase remediation. FIG. 4A illustrates a first acoustic soft surface remediation system 400 that includes an acoustic dislodging mechanism 405 and an acoustic displacing mechanism 410. Acoustic dislodging mechanism 405 and acoustic displacing mechanism 410 are defined similarly as dislodging mechanism 105 and displacing mechanism 110, as described in FIG. 1.

Acoustic dislodging mechanism 405 utilizes acoustic energy (broadly defined, the use of sound to create a wave pattern that travels through air, and stores the energy in the wave pattern) to free contaminants from a soft surface. Varying frequencies and wavelengths, singly or in combination, can be generated by acoustic dislodging mechanism 405. One example of an acoustic dislodging mechanism 405 is an acoustic subwoofer speaker membrane that creates a single low-frequency wavelength that, in turn, dislodges the contaminant from the soft surface.

Acoustic displacing mechanism 410 is defined similarly to acoustic dislodging mechanism 405; however, the nature of parameters such as frequency and wavelength, and wave patterns will be selected to support movement of contaminants away from the soft surface and thus may be quite different from those used for acoustic dislodging mechanism 405.

Additional forms of both process mechanisms: acoustic dislodging mechanism 405 and acoustic displacing mechanism 410 may be suggested easily by those skilled in the art.

In operation, a soft surface is remediated by using acoustic disposing mechanism 405 to deliver sufficient energy to the soft surface to force contaminants to release from the soft surface. Then acoustic displacing mechanism 410 provides the energy to move contaminants away from the soft surface. If desired, another process mechanism needs to be utilized to accomplish the step of disposing, and disinfecting/freshening in first acoustic soft surface remediation system 400. Thus first acoustic soft surface remediation system 400 provides a mechanism to control a variety of contaminants, by simultaneously dislodging and displacing the contaminants, without harming the soft surface.

In a second example shown in FIG. 4B, second acoustic soft surface remediation system 420 includes acoustic dislodging mechanism 422 and acoustic displacing mechanism 424 combined into one apparatus. For example, one waveform generator can create acoustic energy waveforms that accomplish both dislodging and displacing. Thus second acoustic soft surface remediation system 420 can combine both possible process mechanisms in a combined element configuration.

In a third example shown in FIG. 4C, third acoustic soft surface remediation system 430 includes only acoustic dislodging mechanism 432. Examples of acoustic dislodging mechanism 432 are identical to those described in first acoustic soft surface system 300. Thus third acoustic soft surface remediation system 430 can provide for less than all four process mechanisms in its design.

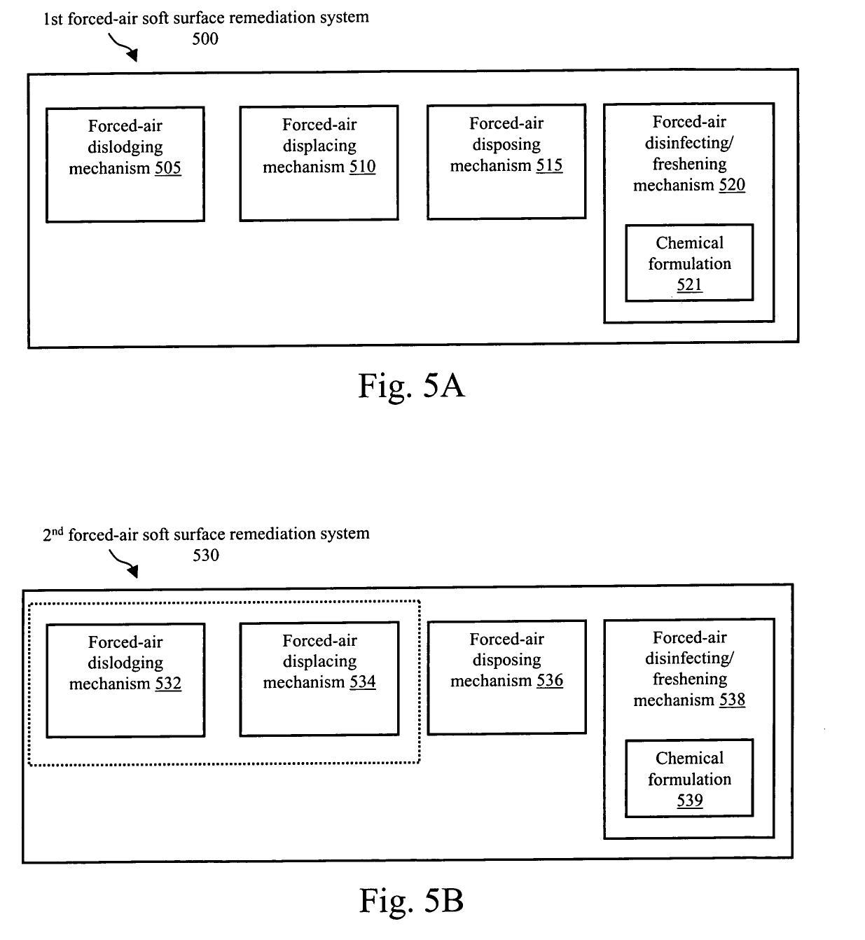

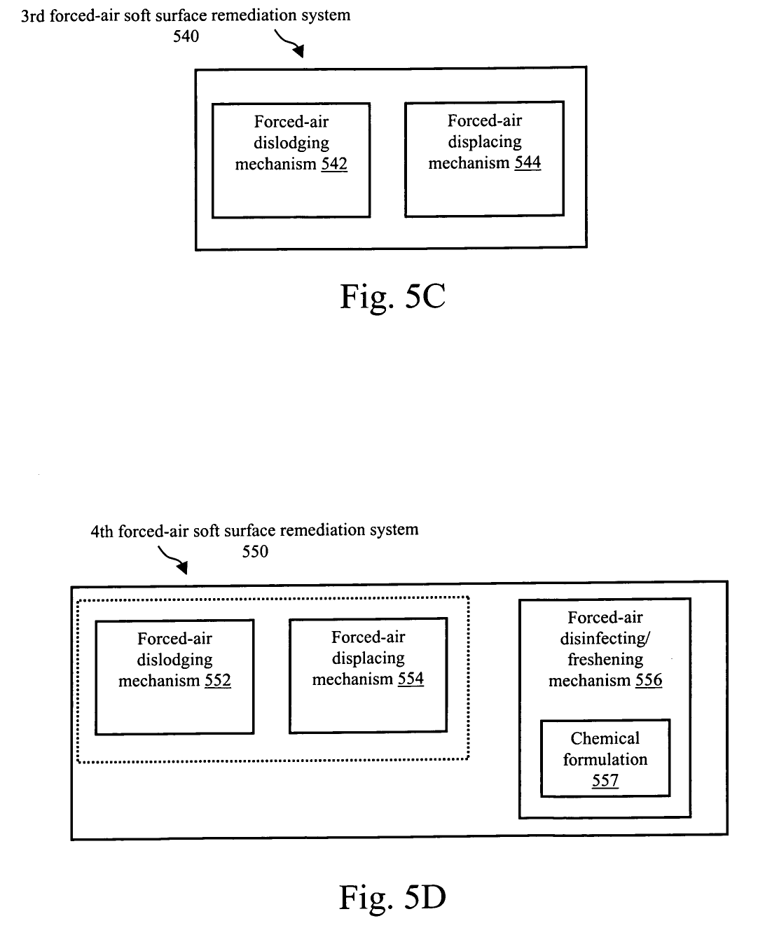

In a fifth embodiment, to accomplish the four phases of dislodging, displacing, disposing of, and disinfecting/freshening, a forced-air based system is provided. FIG. 5A illustrates a forced-air soft surface remediation system 500 that includes a forced-air dislodging mechanism 505, a forced-air displacing mechanism 510, a forced-air disposing mechanism 515, and a forced-air disinfecting/freshening mechanism 520. Forced-air disinfecting/freshening mechanism 520 further includes a chemical formulation 521.

Forced-air dislodging mechanism 505, forced-air displacing mechanism 510, forced-air disposing mechanism 515, and forced-air disinfecting/freshening mechanism 520 are defined similarly as dislodging mechanism 105, displacing mechanism 110, disposing mechanism 115, and disinfecting/freshening mechanism 120, as described in FIG. 1.

Forced-air dislodging mechanism 505 is an air-flow solution that dislodges contaminants, such as generation and application of a stream or jet of air on a soft surface. This is accomplished via tools such as an air fan, a vacuum pump, or a jet stream. Similar tools can be used for forced-air displacing mechanism 510.

Forced-air disposing mechanism 515 utilizes forced-air to direct contaminants into a capture mechanism, such as high-efficiency particulate air (HEPA) or HAF filters, such as those used in a vacuum cleaner, or cyclone cups, cloth filters, and the like. Another example would be the use of a non woven material to scrub the air stream and provide a convenient mechanism to dispose of the contaminants.

Forced-air disinfecting/freshening mechanism 520 utilizes either passive or active delivery of a chemical formulation 521 to the soft surface. An example of passive delivery is an air flow generated to pass through a porous substance (e.g. a gel) that carries particles of chemical formulation 521 away from the porous substance and to the soft surface. An example of an active delivery system is an aerosol-driven, intermittent burst of chemical formulation 521 that may be provided on a timing system or on demand by the user. In either the passive or active scenario, chemical formulation 521 is the chemistry or treatment required for disinfecting/freshening mechanism 520. Chemical formulation 521 can be, in one example, a freshening formula such as Glades Air Freshener, manufactured by SC Johnson & Sons, or a like composition; or in another example, may be a sanitizing formula such as Oust®, also manufactured by SC Johnson & Sons, or a like composition.

Additional forms of all four process mechanisms: forced-air dislodging mechanism 505, forced-air displacing mechanism 510, forced-air disposing mechanism 515, and forced-air disinfecting/freshening mechanism 520 may be suggested easily by those skilled in the art.

In operation, a soft surface is remediated by using forced-air disposing mechanism 505 to deliver sufficient energy to the soft surface to force contaminants to release from the soft surface. Then forced-air displacing mechanism 510 provides the energy to move contaminants away from the soft surface, where forced-air disposing mechanism 515 collects contaminants and precludes the contaminants from being released or resettling upon the soft surface. Finally forced-air disinfecting/freshening mechanism 520 provides energy to kill contaminants (in whole or in part) on a soft.

Thus first forced-air soft surface remediation system 500 provides a mechanism to control a variety of contaminants, by simultaneously dislodging, displacing, disposing of, and disinfecting/freshening the contaminants, without harming the soft surface.

In a second example shown in FIG. 5B, second forced-air soft surface remediation system 530 includes forced-air dislodging mechanism 532, forced-air displacing mechanism 534, forced-air disposing mechanism 536, forced-air disinfecting/freshening mechanism 538, and chemical formulation 539. In this case, forced-air dislodging mechanism 532 is combined with forced-air displacing mechanism 534 into a single apparatus. In one design, second forced-air soft surface remediation system 530 houses a motor apparatus, controls, and fan that perform the function of both forced-air dislodging mechanism 532 and forced-air displacing mechanism 534, i.e. the motor powers the fan such that sufficient cubic feet per minute of airflow is generated to dislodge contaminants and also provides sufficient force to displace and move the contaminants away from the soft surface. A woven filter, or like filtration device through which air flows, acts as forced-air disposing mechanism 536 and a forced-air pump spray device provides the function of forced-air disinfecting/freshening mechanism 538. Thus second forced-air soft surface remediation system 530 can contain all four-process mechanisms in a combined element configuration.

A specific example of this configuration is described in greater detail in application entitled “Soft-Surface Remediation Device And Method Of Using Same” which is U.S. application Ser. No. 11/090,438.

In a third example shown in FIG. 5C, third forced-air soft surface remediation system 540 includes forced-air dislodging mechanism 542, and forced-air displacing mechanism 544. In this case, a forced-air disposing mechanism and disinfecting/freshening mechanism have been omitted and are not included in the design of third forced-air soft surface remediation system 540. Examples of forced-air dislodging mechanism 542 and forced-air displacing mechanism 544 are identical to those described in first forced-air soft surface system 540. Thus third forced-air soft surface remediation system 540 can provide for less than all four-process mechanisms in its design.

In a fourth example shown in FIG. 5D, fourth forced-air soft surface remediation system 550 includes forced-air dislodging mechanism 552, forced-air displacing mechanism 554, and forced-air disinfecting/freshening mechanism 556, further including chemical formulation 557. In this case, a forced-air disposing mechanism has been omitted and is not included in the design of fourth forced-air soft surface remediation system 540, and also forced-air dislodging mechanism 552 is combined with forced-air displacing mechanism 554 into a single apparatus. The examples cited in the second and third examples above also apply to this example. Thus fourth mechanical soft surface remediation system 550 can provide for less than all four-process mechanisms and also combine process mechanisms in its design.

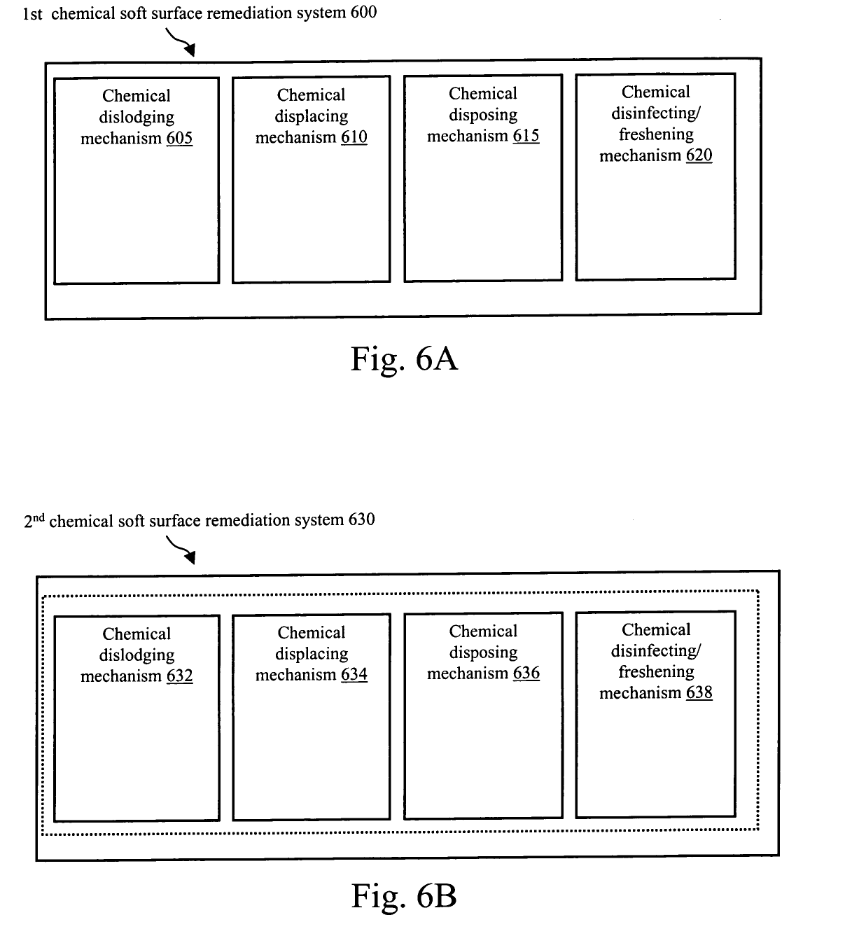

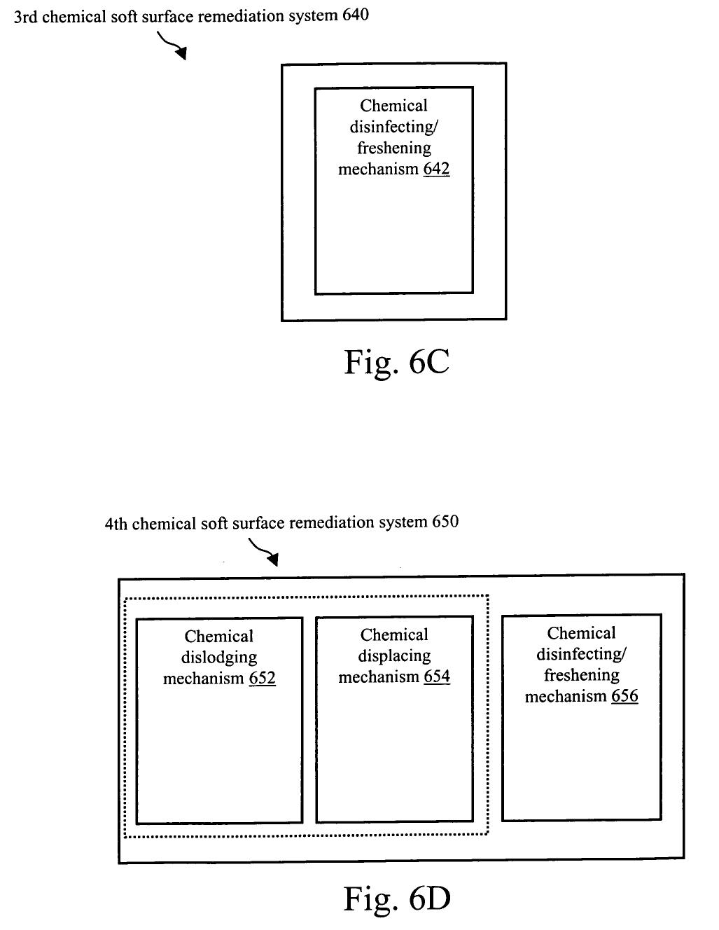

In a sixth embodiment, to accomplish the four phases of dislodging, displacing, disposing of, and disinfecting/freshening, a chemically based system is provided. FIG. 6A illustrates a chemical soft surface remediation system 600 that includes a chemical dislodging mechanism 605, a chemical displacing mechanism 610, a chemical disposing mechanism 615, and a chemical disinfecting/freshening mechanism 620.

Chemical dislodging mechanism 605, chemical displacing mechanism 610, chemical disposing mechanism 615, and chemical disinfecting/freshening mechanism 620 are defined similarly as dislodging mechanism 105, displacing mechanism 110, disposing mechanism 115, and disinfecting/freshening mechanism 120, as described in FIG. 1.

In each of chemical dislodging mechanism 605, chemical displacing mechanism 610, chemical disposing mechanism 615, and chemical disinfecting/freshening mechanism 620, different chemical formulations may be considered for each function. Separately each step can be accomplished via a different composition, that each use the ability of a formulation to change or break the bonding nature of contaminants on a soft surface and further to utilize aqueous chemistry to move and transport contaminants away from a soft surface and contain, dispose, disinfect and freshen. The specific formulation is created or chosen for the desired effect on the soft surface.

Ideally one formulation addresses multiple process mechanisms (see the examples that follow); however it is also feasible for four distinct formulations to be applied separately or be applied using one apparatus that selectively applies each formulation in proper succession.

Additional forms of all four process mechanisms: chemical dislodging mechanism 605, chemical displacing mechanism 610, chemical disposing mechanism 615, and chemical disinfecting/freshening mechanism 620 may be suggested easily by those skilled in the art.

In operation, a soft surface is remediated by using chemical disposing mechanism 605 to deliver sufficient energy to the soft surface to force contaminants to release from the soft surface. Then chemical displacing mechanism 610 provides the energy to move contaminants away from the soft surface, where chemical disposing mechanism 615 collects contaminants and precludes the contaminants from being released or resettling upon the soft surface. Finally, chemical disinfecting/freshening mechanism 620 provides energy to kill contaminants (in whole or in part) on a soft surface.

Thus first chemical soft surface remediation system 600 provides a mechanism to control a variety of contaminants, by simultaneously dislodging, displacing, disposing of, and disinfecting/freshening the contaminants, without harming the soft surface.

In a second example shown in FIG. 6B, second chemical soft surface remediation system 630 includes chemical dislodging mechanism 632, chemical displacing mechanism 634, chemical disposing mechanism 636, chemical disinfecting/freshening mechanism 638. In this case, all four process mechanisms are combined into a single chemical formulation that achieves the steps of dislodging, displacing, disposing, and disinfect/freshening. The single chemical formulation is applied (e.g. sprayed) upon a soft surface and is allowed to act upon the contaminants therein. Thus, second chemical soft surface remediation system 630 can contain all four process mechanisms in a combined element configuration.

In a third example shown in FIG. 6C, third chemical soft surface remediation system 640 only includes chemical disinfecting/freshening mechanism 642. Examples of chemical disinfecting/freshening mechanism 642 are identical to those described in Table 5 above. Thus third chemical soft surface remediation system 640 can provide for less than all four-process mechanisms in its design.

In a fourth example shown in FIG. 6D, fourth chemical soft surface remediation system 650 includes chemical dislodging mechanism 652, chemical displacing mechanism 654, and chemical disinfecting/freshening mechanism 656. In this case, a chemical disposing mechanism has been omitted and is not included in the design of fourth chemical soft surface remediation system 640, and also chemical dislodging mechanism 652 is combined with chemical displacing mechanism 654 into a single formulation. It is feasible that the same chemistry that breaks contaminant bonds to a soft surface also provides displacement away from the soft surface, in one composition. An example includes a chemical that would act like a soft surface surfactant and would detach the contaminant away from the fiber(s) and lift it to the surface where it could be retrieved/recovered and removed. Thus fourth chemical soft surface remediation system 650 can provide for less than all four-process mechanisms and also combine process mechanisms in its design.

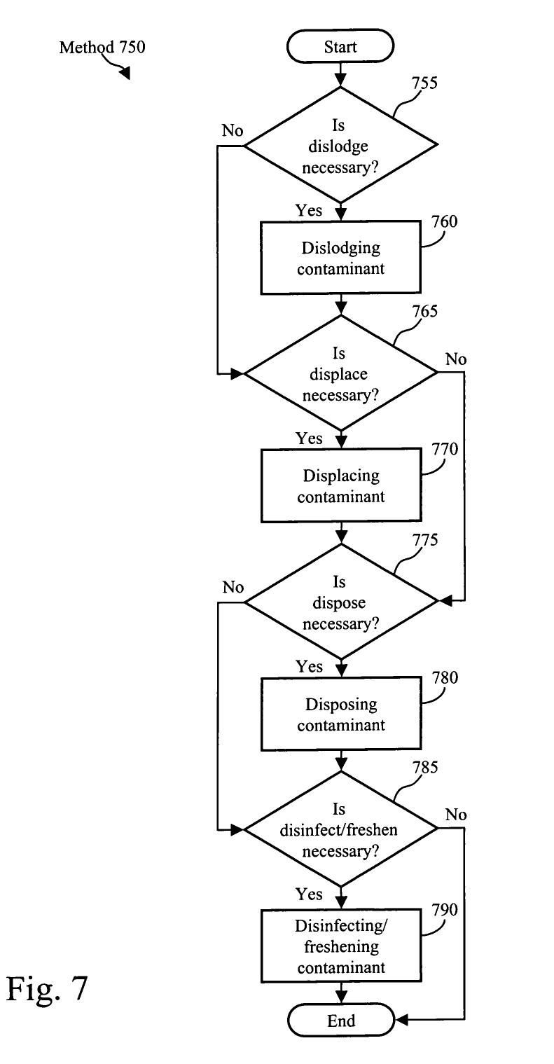

With continuing reference to FIGS. 1, 2, 3, 4, 5, and 6, FIG. 7 illustrates a flow diagram of a method 750 of operating soft surface remediation systems 100, 200, 300, 400, 500, and 600 of the present invention. Method 750 includes the following steps:

In decision step 755, in the case of soft surface remediation system 100, the user determines whether dislodging of contaminants from a soft surface is required. Typically, dislodge is necessary on a surface on which a prior dislodge has not occurred. Generally, the user is a person, such as a homeowner and/or domestic support, who is interested in utilizing the present invention, in order to remediate a soft surface. If yes, method 750 proceeds to step 760; if no, method 750 proceeds to step 765.

Alternatively, in the case of soft surface remediation system 200, the user determines whether mechanical dislodge of contaminants from a soft surface is required. If yes, method 750 proceeds to step 760; if no, method 750 proceeds to step 765.

Alternatively, in the case of soft surface remediation system 300, the user determines whether electrostatic dislodge of contaminants from a soft surface is required. If yes, method 750 proceeds to step 760; if no, method 750 proceeds to step 765.

Alternatively, in the case of soft surface remediation system 400, the user determines whether acoustic dislodge of contaminants from a soft surface is required. If yes, method 750 proceeds to step 760; if no, method 750 proceeds to step 765.

Alternatively, in the case of soft surface remediation system 500, the user determines whether forced-air dislodge of contaminants from a soft surface is required. If yes, method 750 proceeds to step 760; if no, method 750 proceeds to step 765.

Alternatively, in the case of soft surface remediation system 600, the user determines whether chemical dislodge of contaminants from a soft surface is required. If yes, method 750 proceeds to step 760; if no, method 750 proceeds to step 765.

In step 760, in the case of soft surface remediation system 100, the contaminants are dislodged from the soft surface by use of dislodging mechanism 105, which employs one or more of multiple technical mechanisms, such as mechanical, electrostatic, acoustic, forced-air, and chemical. Method 750 proceeds to step 765.

Alternatively, in the case of soft surface remediation system 200, the contaminants are dislodged from the soft surface by use of mechanical dislodging mechanism 205 Method 750 proceeds to step 765.

Alternatively, in the case of soft surface remediation system 300, the contaminants are dislodged from the soft surface by use of electrostatic dislodging mechanism 305. Method 750 proceeds to step 765.

Alternatively, in the case of soft surface remediation system 400, the contaminants are dislodged from the soft surface using acoustic dislodging mechanism 405, which employs acoustic energy mechanism 406 and tool mechanism 407. Method 750 proceeds to step 765.

Alternatively, in the case of soft surface remediation system 500, the contaminants are dislodged from the soft surface by use of forced-air dislodging mechanism 505 Method 750 proceeds to step 765.

Alternatively, in the case of soft surface remediation system 600, the contaminants are dislodged from the soft surface by use of chemical dislodging mechanism 605. Method 750 proceeds to step 765.

In decision step 765, in the case of soft surface remediation system 100, the user determines whether displace of contaminants from a soft surface is required. Typically, displace would be necessary on a surface on which a prior dislodge and/or displace has not occurred. Those skilled in the art will recognize that, sometimes, dislodge and displace can occur simultaneously. If yes, method 750 proceeds to step 770; if no, method 750 proceeds to step 775.

Alternatively, in the case of soft surface remediation system 200, the user determines whether mechanical displace of contaminants from a soft surface is required. If yes, method 750 proceeds to step 770; if no, method 750 proceeds to step 775.

Alternatively, in the case of soft surface remediation system 300, the user determines whether electrostatic displace of contaminants from a soft surface is required. If yes, method 750 proceeds to step 770; if no, method 750 proceeds to step 775.

Alternatively, in the case of soft surface remediation system 400, the user determines whether acoustic displace of contaminants from a soft surface is required. If yes, method 750 proceeds to step 770; if no, method 750 proceeds to step 775.

Alternatively, in the case of soft surface remediation system 500, the user determines whether forced-air displace of contaminants from a soft surface is required. If yes, method 750 proceeds to step 770; if no, method 750 proceeds to step 775.

Alternatively, in the case of soft surface remediation system 600, the user determines whether chemical displace of contaminants from a soft surface is required. If yes, method 750 proceeds to step 770; if no, method 750 proceeds to step 775.

In step 770, in the case of soft surface remediation system 100, the contaminants are displaced from the soft surface by use of displacing mechanism 110, which employs one or more multiple technical mechanisms, such as mechanical, electrostatic, acoustic, forced-air, and chemical. Method 750 proceeds to step 775.

Alternatively, in the case of soft surface remediation system 200, the contaminants are displaced from the soft surface by use of mechanical displacing mechanism 210. Method 750 proceeds to step 775.

Alternatively, in the case of soft surface remediation system 300, the contaminants are displaced from the soft surface using electrostatic displacing mechanism 310. Method 750 proceeds to step 775.

Alternatively, in the case of soft surface remediation system 400, the contaminants are displaced from the soft surface by use of acoustic displacing mechanism 410, which employs acoustic energy mechanism 406 and tool mechanism 407. Method 750 proceeds to step 775.

Alternatively, in the case of soft surface remediation system 500, the contaminants are displaced from the soft surface by use of forced-air displacing mechanism 510. Method 750 proceeds to step 775.

Alternatively, in the case of soft surface remediation system 600, the contaminants are displaced from the soft surface by use of chemical displacing mechanism 610. Method 750 proceeds to step 775.

In decision step 775, in the case of soft surface remediation system 100, the user determines whether dispose of contaminants from a soft surface is required. Typically, dispose would be necessary on a surface on which a prior dislodge and/or displace has occurred. If yes, method 775 proceeds to step 780; if no, method 775 proceeds to step 785.

Alternatively, in the case of soft surface remediation system 200, the user determines whether mechanical dispose of contaminants from a soft surface is required. If yes, method 750 proceeds to step 780; if no, method 750 proceeds to step 785.

Alternatively, in the case of soft surface remediation system 300, the user determines whether electrostatic dispose of contaminants from a soft surface is required. If yes, method 750 proceeds to step 780; if no, method 750 proceeds to step 785.

Alternatively, in the case of soft surface remediation system 400, this method step is not required.

Alternatively, in the case of soft surface remediation system 500, the user determines whether forced-air dispose of contaminants from a soft surface is required. If yes, method 750 proceeds to step 780; if no, method 750 proceeds to step 785.

Alternatively, in the case of soft surface remediation system 600, the user determines whether chemical dispose of contaminants from a soft surface is required. If yes, method 750 proceeds to step 780; if no, method 750 proceeds to step 785.

In this step 780, in the case of soft surface remediation system 100, the contaminants are disposed from the soft surface by use of disposing mechanism 115, which employs one or more multiple technical mechanisms, such as mechanical, electrostatic, acoustic, forced-air, and chemical. Method 750 proceeds to step 785.

Alternatively, in the case of soft surface remediation system 200, the contaminants are mechanically disposed from the soft surface by use of mechanical disposing mechanism 215. Method 750 proceeds to step 785.

Alternatively, in the case of soft surface remediation system 300, the contaminants are electrostatically disposed from the soft surface by use of electrostatic disposing mechanism 315 Method 750 proceeds to step 785

Alternatively, in the case of soft surface remediation system 400, this method step is not required.

Alternatively, in the case of soft surface remediation system 500, the contaminants are disposed from the soft surface by use of forced-air disposing mechanism 515. Method 750 proceeds to step 785.

Alternatively, in the case of soft surface remediation system 600, the contaminants are chemically disposed from the soft surface by use of chemical disposing mechanism 615 Method 750 proceeds to step 785.

In decision step 785, in the case of soft surface remediation system 100, the user determines whether disinfect of contaminants from a soft surface is required. Typically, disinfect is necessary on a surface on which a prior dislodge and/or displace and/or disposal has occurred. If yes, method 750 proceeds to step 790; if no, method 750 ends.

Alternatively, in the case of soft surface remediation system 200, the user determines whether mechanical disinfect of contaminants from a soft surface is required. If yes, method 750 proceeds to step 790; if no, method 750 ends.

Alternatively, in the case of soft surface remediation system 300, this method step is not required.

Alternatively, in the case of soft surface remediation system 400, this method step is not required.

Alternatively, in the case of soft surface remediation system 500, the user determines whether forced-air disinfect of contaminants from a soft surface is required. If yes, method 750 proceeds to step 790; if no, method 750 ends.

Alternatively, in the case of soft surface remediation system 600, the user determines whether chemical disinfect of contaminants from a soft surface is required. If yes, method 750 proceeds to step 790; if no, method 750 ends.

In step 790, in the case of soft surface remediation system 100, the soft surface is disinfected from contaminants by use of disinfecting/freshening mechanism 120. Method 750 ends.

Alternatively, in the case of soft surface remediation system 200, the soft surface is disinfected from contaminants by use of mechanical disinfecting/freshening mechanism 220. Method 750 ends

Alternatively, in the case of soft surface remediation system 300, this method step is not required.

Alternatively, in the case of soft surface remediation system 400, this method step is not required.

Alternatively, in the case of soft surface remediation system 500, the soft surface is disinfected from contaminants by use of forced-air disinfecting/freshening mechanism 520. Method 750 ends.

Alternatively, in the case of soft surface remediation system 600, the soft surface is disinfected from contaminants by use of chemical disinfecting/freshening mechanism 620. Method 750 ends.

As mentioned above, contaminants are of a large variety, and soft surface remediation systems 100, 200, 300, 400, 500, and 600 along with method 750, demonstrate a mechanism used to control the large variety of contaminants.

In a seventh embodiment, it is illustrated that, within one system, numerous combinations of mechanisms for soft surface remediation can be used to accomplish the four phases of dislodging, displacing, disposing, and disinfecting/freshening.

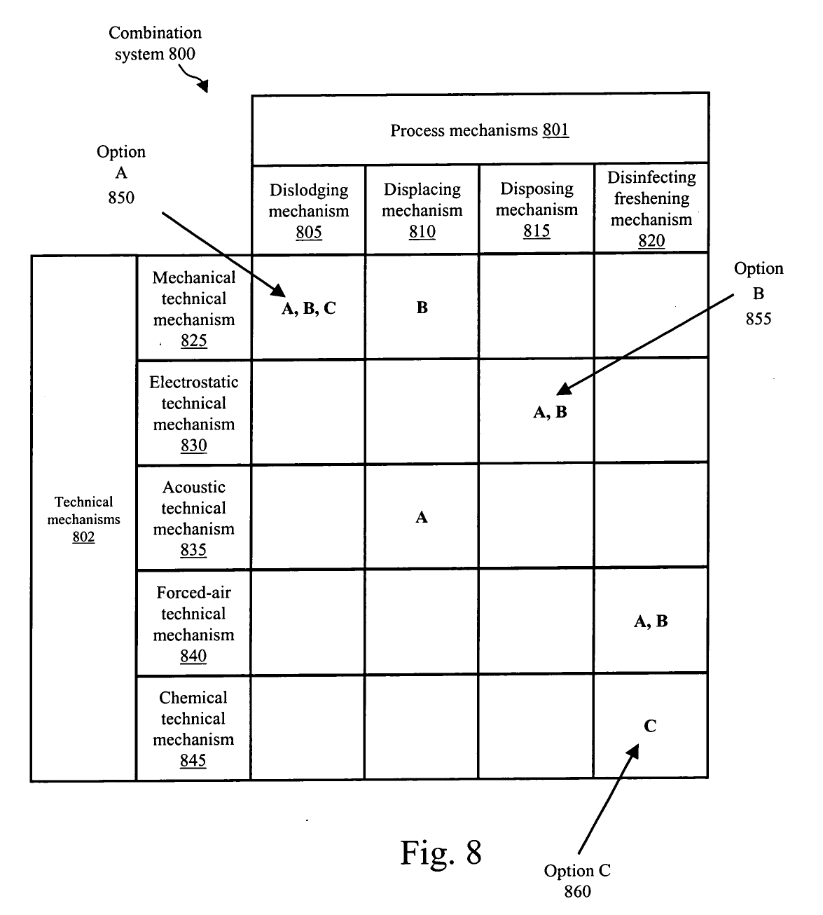

FIG. 8 illustrates a combination system 800 that includes a series of process mechanisms 801, and a series of technical mechanisms 802. Process mechanisms 801 further include dislodging mechanism 805, displacing mechanism 810, disposing mechanism 815, and disinfecting/freshening mechanism 820. Technical mechanisms 802 further include a mechanical technical mechanism 825, an electrostatic technical mechanism 830, an acoustic technical mechanism 835, a forced-air technical mechanism 840, and a chemical technical mechanism 845.

Further shown in FIG. 8 is an option “A” 850, an option “B” 855, and an option “C” 860 within combination system 800. These combinations represent specific selections of process mechanisms 801, and technical mechanisms 802, and are discussed in more detail below.

Process mechanisms 801 represent a series of process steps that are employed to remediate a soft surface. Dislodging mechanism 805, displacing mechanism 810, disposing mechanism 815, and disinfecting/freshening mechanism 820 are defined similarly as dislodging mechanism 105, displacing mechanism 110, disposing mechanism 115, and disinfecting/freshening mechanism 120, as described in FIG. 1. However additional process steps, as identified as necessary for soft surface remediation, could easily be added by those skilled in the art

Technical mechanisms 802 include mechanisms for enabling process mechanisms 801, as has been described in the previous six embodiments.

As illustrated in Table 1 below, it can be seen that each available soft surface remediation mechanism (e.g. mechanical, electrostatic, acoustic, forced-air, chemical) and each step of the four-phases of soft surface remediation provide a variety of combinations between the various technical mechanisms available.

TABLE 1
Exemplary Arrangement of Technical and Process Mechanism
Selections for Four-Phase Soft Surface Remediation

| | Process Mechanisms | | | | |
|---|---|---|---|---|---|
| | | | | | Protect/ |
| Technical | | | | Disinfect/ | Renew |
| Mechanisms | Dislodge | Displace | Dispose | Freshen | (optional) |
| Mechanical | 1 | 0 | 0 | 0 | 0 |
| Mechanism | | | | | |
| Electrostatic | 0 | 1 | 0 | N/A | 0 |
| Mechanism | | | | | |
| Acoustic | 1 | 0 | N/A | N/A | 0 |
| Mechanism | | | | | |
| Forced-air | 0 | 0 | 1 | 0 | 0 |
| Mechanism | | | | | |
| Chemical | 0 | 0 | 0 | 1 | 0 |
| Mechanism | | | | | |
| TOTALS | 2 | 1 | 1 | 1 | 5 |

In Table 1, it can be seen that at least one of each of mechanical, electrostatic, acoustic, forced-air, and chemical technical mechanisms can be selected to accomplish the four phases of process mechanisms dislodge, displace, dispose, and disinfect/freshen/protect/renew. Note that there are certain intersections of Table 1 that are not applicable, e.g. electrostatic mechanisms cannot be used for disinfect/freshening.

However, it can further be extrapolated that numerous arrangements of each technical mechanism are possible. See in Table 2 below for one example of how a technical mechanism (in this case mechanical) may be used in a four-phase soft surface remediation system. In fact, the combinations available are 2ⁿ, where “n” equals the number of process mechanisms addressed in soft surface remediation. In this case for a four-phase soft surface remediation process, there are 2⁴=16 different arrangements where a mechanical mechanism is used in soft surface remediation.

It is also important to note that when one arrangement is selected for a technical mechanism that does not address all four-process mechanisms (e.g. the sixth arrangement shown in Table 2 below), there are multiple possible choice to complete the four-phase soft surface remediation. In the example of the sixth arrangement, there are two more process mechanisms to be completed. When one technical mechanism is selected for fewer than the total number of process mechanisms to be addressed, the remaining process mechanisms are addressed by selecting addition technical mechanism(s), which again provide for multiple choices.

Thus it can be seen that the four process mechanisms of dislodge, displace, dispose, and disinfect/freshen can be covered by any combination possible of the technical mechanisms available. Even if additional technical mechanisms are discovered to be effective in soft surface remediation, the systematic, combined use of these technical mechanisms is within the spirit of the present invention. As can be seen by Table 1, the protect/renew function/mechanism is optional.

TABLE 2
Possible combinations of an exemplary technical mechanism
and the four process mechanisms of dislodge,
displace, dispose, and disinfect/freshen

| Possible Mechanical | | | | |
|---|---|---|---|---|
| Mechanism | Process Mechanisms | | | |
| Arrangements | Dislodge | Display | Dispose | Disinfect/Freshen |
| 1^{st} Arrangement | 0 | 0 | 0 | 0 |
| 2^{nd} Arrangement | 1 | 0 | 0 | 0 |
| 3^{rd} Arrangement | 0 | 1 | 0 | 0 |
| 4^{th} Arrangement | 0 | 0 | 1 | 0 |
| 5^{th} Arrangement | 0 | 0 | 0 | 1 |
| 6^{th} Arrangement | 1 | 1 | 0 | 0 |
| 7^{th} Arrangement | 1 | 0 | 1 | 0 |
| 8^{th} Arrangement | 1 | 0 | 0 | 1 |
| 9^{th} Arrangement | 0 | 1 | 0 | 1 |
| 10^{th} Arrangement | 0 | 1 | 1 | 0 |
| 11^{th} Arrangement | 0 | 0 | 1 | 1 |
| 12^{th} Arrangement | 1 | 1 | 1 | 0 |
| 13^{th} Arrangement | 1 | 0 | 1 | 1 |
| 14^{th} Arrangement | 0 | 1 | 1 | 1 |
| 15^{th} Arrangement | 1 | 1 | 0 | 1 |
| 16^{th} Arrangement | 1 | 1 | 1 | 1 |

Examples of Combination System 800

Option A 850, option B 855, and option C 860 illustrate three examples of how various process mechanisms 801 and technical mechanisms 802 are selected to accomplish soft surface remediation, in order to illustrate the principles of Table 1 and Table 2 above.

First Example (Option A 850): Full Soft-Surface Remediation Accomplished Via One of Each Technical Mechanism

Option A 850 demonstrates multiple process mechanisms 801 can be executed by each of the available technical mechanisms 802. For example, in the case of option A 850, (1) mechanical technical mechanism 825 is used for dislodging mechanism 805, (2) acoustic technical mechanism 830 is used for displacing mechanism 810, (3) electrostatic technical mechanism 835 is used for disposing mechanism 815, and (4) chemical technical mechanism 840 is used for disinfecting/freshening mechanism 820.

Second Example (Option B 855): Selected Process Mechanism for Soft-Surface Remediation Accomplished Via One Technical Mechanism

Option B 855 is an example option, whereby multiple technical mechanisms 802 can be used to execute several process mechanisms 801 and further, that one technical mechanism 802 may execute more than one process mechanism 801. For example, in the case of option B 855, mechanical technical mechanism 825 is used as both dislodging mechanism 805 and displacing mechanism 810. Electrostatic technical mechanism 830 is used for disposing mechanism 815, and forced-air technical mechanism 840 is used for disinfecting/freshening mechanism 820.

Third Example: (Option C 860): Single Selected Process Mechanism for Soft-Surface Remediation Accomplished Via One Technical Mechanism

Option C 860 is an example option, whereby a single technical mechanism 802 is used to execute a single process mechanism and no other activity is required. For example, in the case of option C 860, chemical technical mechanism 845 is used as disinfecting/freshening mechanism 820.

Although the best mode contemplated by the inventors of carrying out the present invention is disclosed above, practice of the present invention is not limited thereto. It will be manifest that various additions, modifications and rearrangements of the features of the present invention may be made without deviating from the spirit and scope of the underlying inventive concept.

In addition, the individual components need not be fabricated from the disclosed materials, but could be fabricated from virtually any suitable materials.

Moreover, the individual components need not be formed in the disclosed shapes, or assembled in the disclosed configuration, but could be provided in virtually any shape, and assembled in virtually any configuration. Further, although may components described herein are physically separate modules, it will be manifest that they may be integrated into the apparatus with which they are associated. Furthermore, all the disclosed features of each disclosed embodiment can be combined with, or substituted for, the disclosed features of every other disclosed embodiment except where such features are mutually exclusive.

It is intended that the appended claims cover all such additions, modifications and rearrangements. Expedient embodiments of the present invention are differentiated by the appended claims.

What is claimed is:

1. A device to control the introduction, generation, and retention of a contaminant on a soft surface comprising:
a dislodging mechanism;
a displacing mechanism operably connected to the dislodging mechanism;
a disposing mechanism in communication with at least one of the dislodging mechanism and displacing mechanism; and
a disinfecting mechanism operably connected to at least one of another mechanism.

2. The device of claim 1 wherein the dislodging mechanism frees a contaminant from or near a soft surface and is a tool that disengages a contaminant from a soft surface by applying sufficient force to overcome the force of adhesion of the contaminant to the soft surface.

3. The device of claim 2 wherein the displacing mechanism moves a contaminant after it has been freed from the soft surface.

4. The device of claim 3 wherein the disposing mechanism as the ability to capture, remove, and dispose of a contaminant after it is freed and moved from the soft surface.

5. The device of claim 1, wherein the disposing mechanism is at least one of: a filter, a non-woven material, a woven material, a contaminant catching cup, a container, sticky tape, fly paper, and a contaminant-attractive gel or liquid.

6. The device of claim 1, wherein the dislodging mechanism is at least one of: a mechanical slapper, an electrostatic attraction material, and an air flow tool.

7. The device of claim 1, wherein the disinfecting mechanism is a treatment for at least one of: dust mites, bacteria, mold, insects, germs, mildew, and refreshening.

8. The device of claim 1, wherein the disinfecting mechanism removes odors or otherwise improves the scent or perceived freshness of the soft surface.

9. The device of claim 1, wherein the disinfecting mechanism includes at least one of: a spray, a liquid, a foam, a powder, and an aerosol to provide at least one of odor removal, biocidial qualities, impairment of organic growth, or positive fragrancing.

10. The device of claim 1, wherein the dislodging mechanism is a chemical formulation that includes at least one of: a gel, a liquid, a foam, and an aerosol driven formulation; and
wherein the disposing mechanism includes a collection tray including an electrostatic mechanism with an electric charge.

11. The device of claim 1, wherein the dislodging mechanism is an acoustic mechanism that includes a device which emits one of a resonant bass tone, sub sonic waveform, sonic waveform, and combination of tones.

12. The device of claim 6, wherein the air flow tool includes an air fan, an air filter in communication with the fan, and a sanitization fluid in operable communication with the fan.

13. The device of claim 1, wherein the dislodging mechanism is at least of: an impact tool, a scraping tool, a brushing tool, an air pressure emitting tool, a slapping tool, a pounding tool, a shaking tool, and a rotating tool.

14. The device of claim 1, wherein the dislodging mechanism has mechanical energy supplied by a wind up device, a battery, or an AC/DC electrical source.

15. The device of claim 1, wherein the disposing mechanism is at least one of: a high energy particulate air filter, filtration device, a cyclone cup, a cloth filter, or high air flow filter; and
wherein the displacing mechanism comprises an electrical charge generator that creates electrical field to repel contaminants from the soft surface.

16. The device of claim 11, wherein the acoustic mechanism includes an acoustic subwoofer speaker membrane that creates a single low frequency wavelength to dislodge contaminants from a soft surface.

17. The device of claim 11, wherein the acoustic mechanism includes a waveform generator that creates the acoustic energy waveforms to accomplish both dislodging and displacing.

18. A system for cleaning comprising:
a low impact remediation technical means for simultaneously dislodging, displacing, disposing of, and disinfecting particulates from a soft surface.

19. The system of claim 18 wherein the technical means is at least one of: an acoustic device, a forced air device, a chemical formula, an electrostatic device, and a mechanical beater.

20. A method of soft surface remediation comprising the steps of:
dislodging particulates from a surface,
displacing the particulates,
disposing of the displaced particulates, and
disinfecting the surface to protect from reintroduction of other particulates.
